(19) United States
(12) Patent Application Publication
(10) Pub. No.: US 2018/0320136 A1
(43) Pub. Date: Nov. 8, 2018

(54) METHODS AND COMPOSITIONS FOR THE PRODUCTION OF MONOCLONAL ANTIBODIES, HEMATOPOIETIC STEM CELLS, AND METHODS OF USING THE SAME

(71) Applicant: GlycoMimitics, Inc., Rockville, MD (US)

(72) Inventor: John L. MAGNANI, Gaithersburg, MD (US)

(73) Assignee: GlycoMimitics, Inc., Rockville, MD (US)

(21) Appl. No.: 15/773,086

(22) PCT Filed: Nov. 2, 2016
(86) PCT No.: PCT/US2016/060014

§ 371 Date: May 2, 2018

Related U.S. Application Data
(60) Provisional application No. 62/250,424, filed on Nov. 3, 2015.

(51) Int. Cl. C12N 5/0789; C07K 16/28

(52) U.S. Cl. CPC C12N 5/0647; C07K 16/2896

(57) ABSTRACT

Methods and compositions for the discovery and production of antibodies that can be used to identify and/or isolate hematopoietic stem cells (HSCs), for example, HSCs with high reconstitution potential. Methods and compositions are further provided for the treatment of patients with hematologic or genetic disorders, patients with cardiovascular disorders, patients recovering from wounds, or patients recovering from chemotherapy or radiation exposure using HSCs or genetically modified HSCs, for example, HSCs and/or genetically modified HSCs with high reconstitution potential.

This application claims the benefit under 35 U.S.C. § 119(e) to U.S. Provisional Application No. 62/250,424 filed Nov. 3, 2015, which application is incorporated by reference herein in its entirety.

The disclosure generally relates to the field of biochemistry, molecular biology, cell biology, and regenerative medicine. The present disclosure provides methods and compositions for the discovery and production of antibodies that can be used to identify and isolate hematopoietic stem cells (HSCs), for example, HSCs with high reconstitution potential. The present disclosure further provides methods and compositions for the treatment of patients with hematologic diseases, disorders, or conditions or patients recovering from chemotherapy or radiation therapy using hematopoietic stem cells, for example, HSCs with high reconstitution potential. The present disclosure further provides methods and compositions for the use of HSCs in the treatment of cardiovascular disorders. The present disclosure further provides methods and compositions for the use of HSCs in the treatment of wounds and to promote wound healing. The present disclosure further provides methods and compositions for the use of HSCs in gene therapy.

The following is provided as background information only and should not be taken as an admission that any subject matter discussed or that any reference mentioned is prior art to the instant disclosure. All publications and patent applications herein mentioned are incorporated by reference to the same extent as if each individual publication or patent application was specifically and individually indicated to be incorporated by reference.

Hematopoietic stem cells (HSCs) are single cells that have a life-long ability to self-renew and to make all blood cell lineages. HSCs play an important role in successful hematopoietic reconstitution using both autologous and allogeneic hematopoietic cell transplants. Weissman I L, Shizuru J A (2008) The origins of the identification and isolation of hematopoietic stem cells, and their capability to induce donor-specific transplantation tolerance and treat autoimmune diseases, Blood 112(9):3543-53, which is hereby incorporated by reference, Hematopoietic cell transplantation is a promising therapeutic approach in the treatment of hematologic diseases, disorders, or conditions (e.g., thalassemias, sickle cell disease, leukemias, lymphomas, myelomas) as well as in rescue from chemotherapy and high-dose radiation. A potential drawback of hematopoietic cell transplants is that they contain a mixture of hematopoietic cells, including HSCs of varying reconstitution potential and other non-HSCs. This may lead to complications and side effects from the procedure. For example, because HSCs with high reconstitution potential cannot be isolated from a patient's other potentially diseased cells, autologous hematopoietic cell transplantations are normally not a treatment option. Instead, allogenic donations are used. By using allogenic cells, a patient may avoid reintroducing diseased cells from his own autologous donation into his system, but risks such complications as graft-versus-host disease. Even in allogenic donations, complications could be reduced by identifying and transplanting only the cells most likely to reconstitute a patient's healthy blood cell populations. Thus, there exists a need to identify and isolate HSCs—especially HSCs with high reconstitution potential—for use in treatments such as hematopoietic cell transplants.

Genetically modified HSCs may also provide a promising therapeutic approach in the treatment of genetic diseases, disorders, or conditions, especially hematologic genetic diseases, disorders, or conditions. Because of HSCs unique characteristics (e.g., the ability to self-renew and differentiate into numerous cells), they are ideal candidates for introducing genetic fixes into a patient. In gene therapy, cells obtained from a patient suffering from a genetic disease, disorder, or condition are genetically modified using known techniques to insert the therapeutic gene(s) (either integrated into the host cell's genomes or as external episomes or plasmids). The therapeutic gene may, for example, cause the cell to express proteins, interfere with protein expression, or correct a genetic mutation. A common method of gene therapy involves using a vector to insert a polymer, such as DNA, into a genome, thereby replacing a mutated or otherwise dysfunctional gene with the functional therapeutic gene. The cells with genetic modifications could then be transplanted back into to the patient, and the genetically modified cells would express the therapeutic gene(s), thereby treating the disease, disorder, or condition. The effectiveness of such gene therapies is related to how primitive the genetically modified cells are. Drawbacks of current methods may relate to the rapidly dividing nature and short life-spans of many cells, which prevent the gene therapies from achieving long-term benefits and/or require patients to undergo multiple treatments.

HSCs, for example, HSCs with high reconstitution potential, can differentiate into endothelial progenitor cells (EPCs). Several studies report that EPCs promote neovascularization and re-endothelialization, which correlate to the healing of cardiovascular disorders and wound recovery. See, e.g., Krankel, N., et al., “‘Endothelial progenitor cells’ as a therapeutic strategy in cardiovascular disease,” Curr, Vase. Pharmacol., January, 2012, Vol. 10(1):107-24, which is hereby incorporated by reference. EPCs derived from HSCs may, for example, be administered to a patient to treat cardiovascular disorders or to treat wounds (e.g., to promote wound healing) and/or be administered to a patient to differentiate in vivo into EPCs. EPCs derived from HSCs may, for example, also be used to condition serum or other media so that the conditioned media may be administered to the patient. But, as noted above, ways to identify HSCs, for example, HSCs with high reconstitution potential, in order to obtain EPCs—without directly testing the HSCs' reconstitution abilities in in vivo assays—has previously been unknown.

HSCs, for example, HSCs with high reconstitution potential, are better candidates for gene therapies because they are more likely to produce therapeutic results and/or produce more favorable (faster, longer-lasting, more robust) therapeutic results. Thus, there exists a need to identify and isolate HSCs—especially HSCs with high reconstitution potential—for use in gene therapies. HSCs can be isolated from various sources, including bone marrow, mobilized peripheral blood, and cord blood. The process of identifying and isolating the HSC population from a cell mix can involve the use of HSC-specific markers, such as CD34, and in vivo assays, such as rescue of lethally irradiated mice with limiting doses of candidate HSCs. The ability of HSCs to reconstitute blood cell lines varies, however, and HSC-specific markers only identify HSCs but cannot identify the subpopulation of HSCs with high reconstitution potential. Ways to identify HSCs with high reconstitution potential—without directly testing their reconstitution abilities in in vivo assays—has previously been unknown.

Furthermore, it was previously thought that all HSCs expressed the CD34 marker. But there is evidence that certain HSCs do not express CD34. E.g., Nakauchi, Hirmitsu, Hematopoietic stem cells: Are they CD34-positive or CD34-negative, Nature medicine 4:1009-1010 (1998), which is hereby incorporated by reference. Thus, there exists a need to identify CD34-negative HSCs.

Efforts to fully exploit HSCs potential for clinical purposes have been hampered by the limited knowledge regarding markers that specifically and effectively allow for HSC isolation and expansion. In particular, it was previously unknown how to identify HSCs with high reconstitution potential absent in vivo assays.

The following is provided as a means to address one or more of these needs.

To assist in the detection and isolation of HSCs, new markers were identified in the surface of these cells. These markers comprise 2-3 sialylated lacto-neolacto-type structures (e.g., sialyl I, sialyl i, sialyllactose, sialyllacto-N-tetraose, sialyllacto-N-neotetraose, N-acetyl sialyllactoseamine). These structures are present in the surface of primitive early hematopoietic stem cells, for example, HSCs with high reconstitution potential, such as, for example, in the human stem cell marker CD133. They are expressed predominantly on stem cells and become fucosylated on progenitor cells.

In accordance with the disclosure, methods and compositions are provided for the discovery and production of antibodies that can be used to identify and isolate primitive early hematopoietic stem cells, for example, HSCs with high reconstitution potential. In some instances, the antibodies bind to one or more of the new markers identified herein. A number of applications in regenerative medicine for both the cells isolated by such methods and the antibodies generated by the same are also provided.

In one embodiment, the technologies disclosed herein provide new strategies for the rapid development of diagnostic and therapeutic antibodies for the detection and isolation of primitive early HSCs, for example, HSCs with high reconstitution potential, and treatment by transplantation of diseases, disorders, or conditions of the blood, including thalassemias, sickle cell disease, leukemias, lymphomas, and myelomas. In one embodiment, the HSCs or antibodies are used for treating advanced follicular lymphoma. In another embodiment, the HSCs or antibodies are used for treatment of a pediatric hematologic disease. In one embodiment, the HSCs or antibodies are used for treating an adult hematologic disease. In another embodiment, the HSCs or antibodies are used to treat acute myeloid leukemia. In one embodiment, the HSCs are from a donor that is different from the patient (i.e., allogenic donation). In one embodiment, the donor of the HSCs and the patient are the same person (i.e., autologous donation).

In one embodiment, the technologies disclosed herein provide new strategies for the rapid development of diagnostic and therapeutic antibodies for the detection and isolation of primitive early HSCs, for example, HSCs with high reconstitution potential, and treatment of cardiovascular disease or treatment of wounds (e.g., promotion of wound healing) by administration of the HSCs, EPCs derived from HSCs, or media conditioned by EPCs derived from HSCs. In one embodiment, the HSCs are from a donor that is different from the patient (i.e., allogenic donation). In one embodiment, the donor of the HSCs and the patient are the same person (i.e., autologous donation).

In one embodiment, the technologies disclosed herein provide new strategies for the rapid development of diagnostic and therapeutic antibodies for the detection and isolation of primitive early HSCs, for example, HSCs with high reconstitution potential, for genetic modification and for the treatment of genetic diseases, disorders or conditions, for example, hematological genetic diseases, disorders or conditions, by transplantation of genetically modified HSCs. In one embodiment, the genetically modified HSCs are used to treat diseases or disorders of the blood such as sickle cell, thalassemia, or severe combined immune deficiency.

In one embodiment, a novel approach is provided for the discovery and production of antibodies that can be used to identify and isolate primitive early hematopoietic stem cells with high reconstitution potential, the method comprising immunizing animals with HSCs and then selecting those animals, or cells from those animals, for the presence or production of antibodies that bind 2-3 sialylated lacto-neolacto-type structures (e.g., sialyl I, sialyl i, sialyllactose sialyllacto-N-tetraose, sialyllacto-N-neotetraose, N-acetyl sialyllactoseamine). In some embodiments, the animals are immunized with HSCs that are CD34-positive. In another embodiment, the antibody is selected for binding to 2-3 sialylated lacto-neolacto-type structures (e.g., sialyl I, sialyl i, sialyllactose, sialyllacto-N-tetraose, sialyllacto-N-neotetraose, N-acetyl sialyllactoseamine) on the surface of CD133.

In another embodiment, the method comprises immunizing animals with CD133 isolated from HSCs and then selecting those animals, or cells from those, animals, for the presence or production of antibodies that bind 2-3 sialylated lacto-neolacto-type structures (e.g., sialyl I (big I), sialyl i (small i), sialyllactose). In some embodiments, the CD133 is from CD34 positive (CD34⁺) HSCs. In another embodiment, the antibody is selected for binding to 2-3 sialylated lacto-neolacto-type structures (e.g., sialyl I (big I), sialyl i (small i), sialyllactose) on the surface of CD133.

In one embodiment, any other method (e.g., phage display) capable of generating or providing a panel of antibodies that can be screened for those that bind 2-3 sialylated lacto-neolacto-type structures (e.g., sialyl I (big I), sialyl i (small i), sialyllactose) can be used as a source of antibodies.

In one embodiment, the immunized animals are mice, rats, rabbits, or another mammal. In other embodiments, such antibodies can be generated in non-human transgenic animals, e.g., as described in PCT App. Pub. Nos. WO 01/14424 and WO 00/37504, which are hereby incorporated by reference.

In one embodiment, the antibody is a monoclonal antibody (mAb), which is a substantially homogeneous population of antibodies to a specific antigen. MAbs may be obtained by methods known to those skilled in the art. See, for example Kohler et al. (1975); U.S. Pat. No. 4,376,110; Ausubel et al. (1987-1999); Harlow et al. (1988); and Colligan et al. (1993), which is hereby incorporated by reference. The mAbs envisioned herein may be of any immunoglobulin class including IgG, IgM, IgE, IgA, and any subclass thereof. A hybridoma producing a mAb may be cultivated in vitro or in vivo. High titers of mAbs can be obtained through in vivo production where cells from the individual hybridomas are injected intraperitoneally into pristine-primed Balb/c mice to produce ascites fluid containing high concentrations of the desired mAbs. MAbs of isotype IgM or IgG, for example, may be purified from such ascites fluids, or from culture supernatants, using column chromatography methods or any other methods well-known to those of skill in the art.

In one embodiment, the antibodies are chimeric antibodies. Chimeric antibodies are molecules, different portions of which are derived from different animal species, such as those having a variable region derived from a murine mAb and a human immunoglobulin constant region. Antibodies that have variable region framework residues substantially from human antibody (termed an acceptor antibody) and complementarity determining regions substantially from a mouse antibody (termed a donor antibody) are also referred to as humanized antibodies. Chimeric antibodies are primarily used to reduce immunogenicity in application and to increase yields in production, for example, where murine mAbs have higher yields from hybridomas but higher immunogenicity in humans, such that human/murine chimeric mAbs are used. Chimeric antibodies and methods for their production are known in the art (see, e.g., Better et al., 1988, Cabilly et al., 1984, Harlow et al., 1988; Liu et al, 1987; Morrison et al., 1984; Boulianne et al., 1984; Neuberger et al., 1985; Sahagan et al., 1986; Sun et al., 1987; Cabilly et al., European Patent Applications 125023, 171496, 173494, 184187, 173494, PCT patent applications WO 86/01533, WO 97/02671. WO 90/07861, WO 92/22653 and U.S. Pat. Nos. 5,693,762, 5,693,761, 5,585,089, 5,530,101 and 5,225,539).

In one embodiment, the antibodies are humanized antibodies. In some embodiments, the humanized antibodies comprise both human heavy and light constant domains. In some embodiments, humanized antibodies retain a significant proportion of the binding properties of the parent antibody, which can be, for example, a mouse monoclonal antibody. The humanized antibodies described herein are produced by the intervention of man. Thus, they are not expected to occur n nature. In one embodiment, the humanized antibodies are prepared by techniques well-known in the art, such as those described in Antibody Engineering, Second Edition, Edited by Roland Kontermann and Stefan Dübel and references cited therein.

In one embodiment, a population of cells is provided for the discovery and production of antibodies that can be used to identify and isolate hematopoietic stem cells, for example, HSCs with high reconstitution potential. In one embodiment, the HSCs are used for the production of recombinant antibodies binding to 2-3 sialylated lacto-neolacto-type structures (e.g., sialyl I (big I), sialyl i (small i), sialyllactose). In one embodiment, the recombinant antibodies bind to 2-3 sialylated lacto-neolacto-type structures (e.g., sialyl I (big I), sialyl i (small i), sialyllactose) on the surface of CD133.

In one embodiment, the antibodies produced according to one of the methods described herein have specificity for 2-3 sialylated lacto-neolacto-type structures (e.g., sialyl I (big I), sialyl i (small i), sialyllactose). In one embodiment, the antibodies produced according to one of the methods described herein bind to 3′sialyllactosaminylated-CD133 and not to neuraminidase-treated 3′sialylyllactosaminylated-CD133. In one embodiment, the antibody binds to CD133 and human-fucosidase-treated-CD133 but does not bind to neuraminidase-treated CD133.

In one embodiment, a method is provided for the production of stem cells, the method comprising isolating hematopoietic stem cells, for example, HSCs with high reconstitution potential, and then expanding them in vitro.

In one embodiment, a method is provided for the production of genetically modified stem cells, the method comprising isolating hematopoietic stem cells, for example, HSCs with high reconstitution potential, genetically modifying the HSCs, and then expanding the genetically modified HSCs in vitro.

In one embodiment, a composition is provided comprising HSCs identified according to one of the methods of the disclosure, stem cells derived from the HSCs according to one of the methods of the disclosure, and/or partially or fully differentiated cells propagated from HSCs according to one of the methods of the disclosure. In one embodiment, a composition is provided comprising EPCs propagated from the HSCs. In one embodiment, a composition is provided comprising media conditioned by EPCs propagated from the HSCs.

In one embodiment, a composition is provided comprising HSCs identified according to one of the methods of the disclosure and genetically modified for use in gene therapies, genetically modified stem cells derived from the genetically modified HSCs according to one of the methods of the disclosure, and/or partially or fully differentiated genetically modified cells propagated from genetically modified HSCs according to one of the methods of the disclosure.

In another embodiment, the HSCs identified according to one of the methods of the disclosure, the stem cells produced according to one of the methods of the disclosure, and/or partially or fully differentiated cells propagated from HSCs according to one of the methods of the disclosure may be used directly in therapeutic strategies for treating a variety of disease states and conditions, including in the treatment of hematologic diseases, disorders or conditions (e.g., thalassemias, sickle cell disease, leukemias, lymphomas, myelomas) as well as in rescue from chemotherapy and high-dose radiation. In one embodiment, the HSCs may be used directly in therapeutic strategies for treating cardiovascular disorders. In one embodiment, HSCs may be used directly in therapeutic strategies for treating wounds (e.g., promoting wound healing). In one embodiment, EPCs propagated from HSCs may be used directly in therapeutic strategies for treating cardiovascular disorders. In one embodiment, EPCs propagated from HSCs may be used directly in therapeutic strategies for treating a wound (e.g., promoting wound healing). In one embodiment, EPCs propagated from HSCs may be used to condition media that can be used in therapeutic strategies for treating cardiovascular disorders, treating wounds, or promoting wound healing.

In another embodiment, the HSCs identified and genetically modified according to one of the methods of the disclosure, the genetically modified stem cells produced according to one of the methods of the disclosure, and/or partially or fully differentiated genetically modified cells propagated from genetically HSCs according to one of the methods of the disclosure may be used directly in therapeutic strategies for treating a variety of disease states and conditions, including in the treatment of genetic diseases, disorders or conditions (e.g., sickle cell, thalassemia, or severe combined immune deficiency).

In another embodiment, the stem cells produced according to one of the methods of the disclosure may be partially or full differentiated into cells of a desired lineage and those differentiated cells may be used in therapeutic strategies for treating a variety of disease states and conditions, including in the treatment of hematologic diseases, disorders or conditions (e.g., thalassemias, sickle cell disease, leukemias, lymphomas, myelomas) as well as in rescue from chemotherapy and high-dose radiation. In another embodiment, the stem cells produced according to one of the methods of the disclosure may be partially or fully differentiated into EPCs which may be used in therapeutic strategies for treating cardiovascular disorders, treating wounds, or promoting wound healing.

In another embodiment, the genetically modified stem cells produced according to one of the methods of the disclosure may be partially or fully differentiated into genetically modified cells of a desired lineage and those differentiated cells may be used in therapeutic strategies for treating a variety of disease states and conditions, including in the treatment of genetic diseases, disorders or conditions (e.g., sickle cell, thalassemia, or severe combined immune deficiency).

Additional objects and advantages of the disclosure will be set forth in part in the description which follows, and in part will be obvious from the description, or may be learned by practice of the disclosure. Some of the objects and advantages of the disclosure will be realized and attained by means of the elements and combinations particularly pointed out in the appended claims.

It is to be understood that both the foregoing general description and the following detailed description are exemplary and explanatory only and are not restrictive of the disclosure, as claimed.

### BRIEF DESCRIPTION OF THE DRAWINGS

FIG. 1a depicts the chemical structure of sialyl I (big I).

FIG. 1b depicts the chemical structure of sialyl I (small i).

FIG. 2a depicts the chemical structure of N-acetyl sialyllactoseamine

FIG. 2b depicts the chemical structure of sialyllacto-N-tetraose

FIG. 2c depicts the chemical structure of sialyllacto-N-neotetraose

FIG. 3 depicts the chemical structure of sialyllactose.

Reference will now be made in detail to the present embodiments (exemplary embodiments) of the disclosure, examples of which are illustrated in the accompanying drawings. Wherever possible, the same reference numbers will be used throughout the drawings to refer to the same or like parts.

The abbreviations used herein generally have their conventional meaning in the chemical and biological arts.

The term “antibody,” “antibodies,” “ab,” or “immunoglobulin” are used interchangeably in the broadest sense and include monoclonal antibodies, including isolated, engineered, chemically synthesized or recombinant antibodies (e.g., full length or intact monoclonal antibodies) and also antibody fragments, so long as they exhibit the desired biological activity. In one embodiment, the disclosure relates to monoclonal antibodies.

An antibody molecule consists of a glycoprotein comprising at least two heavy (H) chains and two light (L) chains inter-connected by disulfide bonds. Each heavy chain comprises a heavy chain variable region (or domain) (abbreviated herein as HCVR or VH) and a heavy chain constant region. The heavy chain constant region comprises three or four domains, CH1, CH2, CH3, and CH4. Each light chain comprises a light chain variable region (abbreviated herein as LCVR or VL) and a light chain constant region. The light chain constant region comprises one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g. effector cells) and the first component (C1q) of the classical complement system.

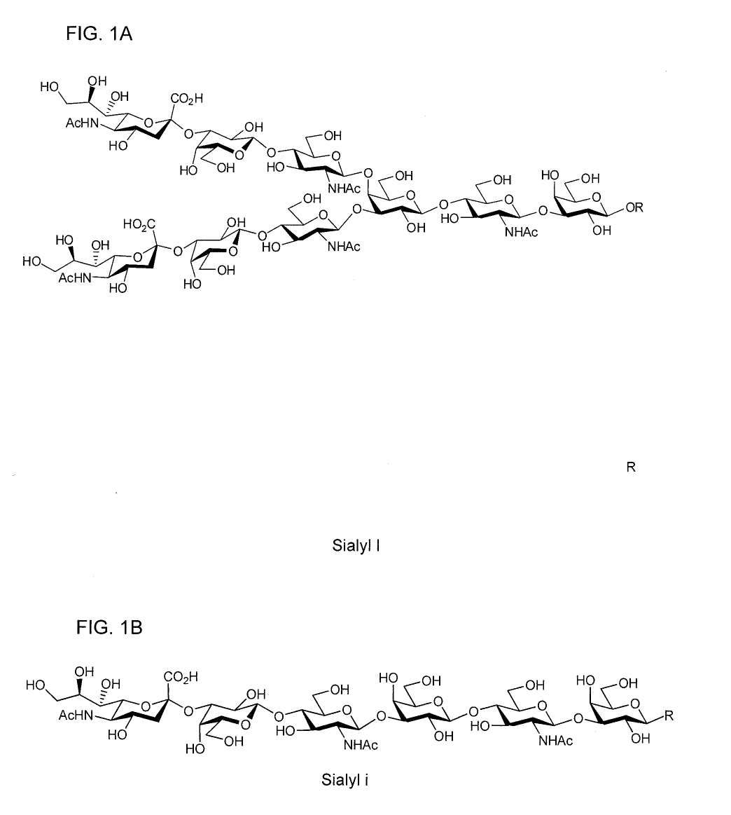

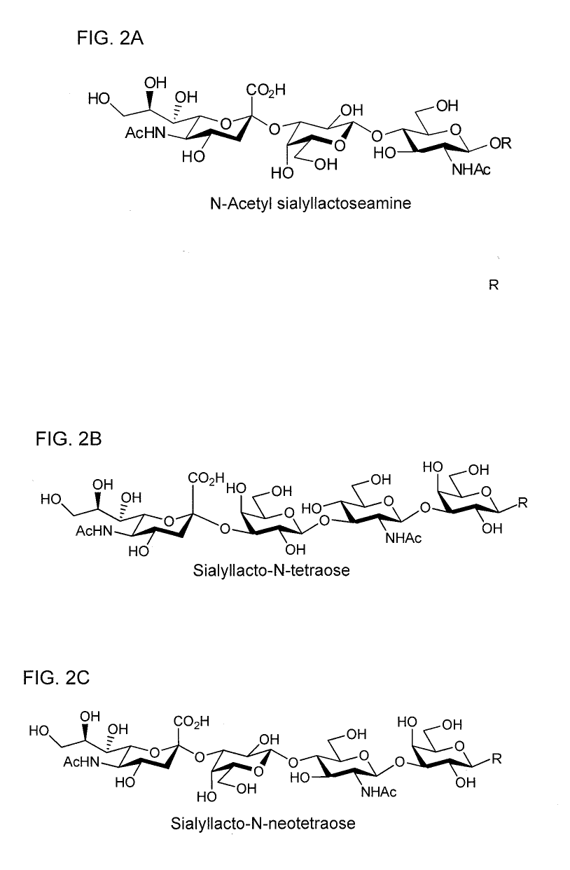

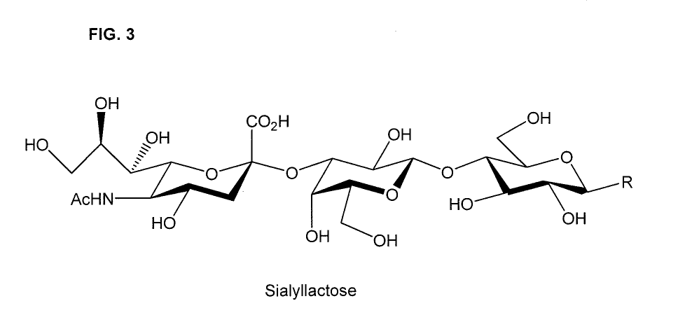

By “antigen binding fragment” of an antibody according to the disclosure, it is intended to indicate any peptide, polypeptide, or protein retaining the ability to bind to the target of the antibody. In one embodiment, the target is selected from 2-3 sialylated lacto-neolacto type structures, such as sialyl I (big I, see FIG. 1a), sialyl i (small i, see FIG. 1b), N-acetyl sialyllactoseamine (see FIG. 2a), sialyllacto-N-tetraose (see FIG. 2b), sialyllacto-N-neotetraose (see FIG. 2c), and sialyllactose (see FIG. 3). In certain embodiments, antigen binding fragments are produced by recombinant DNA techniques. In additional embodiments, binding fragments are produced by enzymatic or chemical cleavage of intact antibodies. Binding fragments include, but are not limited to, Fab, Fab′, F(ab′)₂, Fv, and single-chain antibodies.

The term “monoclonal antibody” or “Mab” as used herein refers to an antibody obtained from a population of substantially homogeneous antibodies, i.e. the individual antibodies of the population are identical except for possible naturally occurring mutations that may be present in minor amounts. Typically, monoclonal antibodies are highly specific, being directed against a single epitope. Such a monoclonal antibody can be produced by a single clone of B cells or hybridoma. Monoclonal antibodies can also be recombinant, i.e., produced by protein engineering. Monoclonal antibodies can also be isolated from phage antibody libraries. In addition, in contrast with preparations of polyclonal antibodies which typically include various antibodies directed against various determinants, or epitopes, each monoclonal antibody is directed against a single epitope of the antigen. The disclosure relates to an antibody isolated or obtained by purification from cells or obtained by genetic recombination or chemical synthesis.

The term “antigen” refers to a molecule or a portion of a molecule capable of being bound by a selective binding agent, such as an antibody, and additionally capable of being used in an animal to produce antibodies capable of binding to an epitope of that antigen. An antigen may have one or more epitopes. Examples of antigens include the 2-3 sialylated lacto-neolacto type structures on the surface of the CD133 molecule (e.g., 3′SL-CD133), such as sialyl I (big I, see FIG. 1a), sialyl i (small i, see FIG. 1b), N-acetyl sialyllactoseamine (see FIG. 2a), sialyllacto-N-tetraose (see FIG. 2b), sialyllacto-N-neotetraose (see FIG. 2c), and sialyllactose (see FIG. 3).

The term “epitope” includes any determinant, such as, for example, a polypeptide determinant, capable of specific binding to an immunoglobulin or T-cell receptor. In certain embodiments, epitope determinants include chemically active surface groupings of molecules such as amino acids, sugar side chains, phosphoryl, or sulfonyl, and, in certain embodiments, may have specific three-dimensional structural characteristics, and/or specific charge characteristics. An epitope is a region of an antigen that is bound by an antibody. In certain embodiments, an antibody is said to specifically bind an antigen when it preferentially recognizes its target antigen in a complex mixture of proteins and/or macromolecules. In one embodiment, an antibody is said to specifically bind an antigen when the dissociation constant is less than or equal to about 1 μM, such as, for example, when the dissociation constant is less than or equal to about 100 nM, such as, for example, when the dissociation constant is less than or equal to about 1 nM, and such as, further for example, when the dissociation constant is less than or equal to about 100 pM. The terms “specific for” and “specific binding,” as used herein, are interchangeable and refer to antibody binding to a predetermined antigen, e.g., the 2-3 sialylated lacto-neolacto type structures such as sialyl I (big I, see FIG. 1a), sialyl i (small i, see FIG. 1b), N-acetyl sialyllactoseamine (see FIG. 2a), sialyllacto-N-tetraose (see FIG. 2b), sialyllacto-N-neotetraose (see FIG. 2c), and sialyllactose (see FIG. 3). Typically, the antibody binds with a dissociation constant (KD) of 10⁻⁶ M or less, and binds to the predetermined antigen with a KD that is at least twofold less than its KD for binding to a nonspecific antigen (e.g., BSA, casein, or any other specified polypeptide) other than the predetermined antigen. The phrases “an antibody recognizing an antigen” and “an antibody specific for an antigen” are used interchangeably herein with the term “an antibody which binds specifically to an antigen.”

The term “stem cells,” as used herein, refers to cells capable of differentiation into other cell types, including those having a particular, specialized function (i.e., terminally differentiated cells, such as erythrocytes and macrophages). Stem cells can be defined according to their source (adult/somatic stem cells, embryonic stem cells), or according to their potency (totipotent, pluripotent, multipotent and unipotent).

The term “hematopoietic stem cells” or “HSCs” refers to animal cells, for example mammalian (including human) cells, that have the ability to self-renew and to differentiate into any of several types of blood cells, including red blood cells and white blood cells, including lymphoid cells and myeloid cells. HSCs can also differentiate into EPCs. HSCs can include hematopoietic cells having long-term engrafting potential in vivo. Long term engrafting potential (e.g., long term hematopoietic stem cells) can be determined using animal models or in vitro models. Animal models for long-term engrafting potential of candidate human hematopoietic stem cell populations include the SCID-hu bone model (Kyoizumi et al, (1992) Blood 79:1704; Murray et al., (1995) Blood 85(2) 368-378) and the in utero sheep model (Zanjani et al., (1992) J. Clin. Invest. 89:1179). For a review of animal models of human hematopoiesis, see Srour et al., (1992) J, Hematother. 1:143-153 and the references cited therein. An in vitro model for stem cells is the long-term culture-initiating cell (LTCIC) assay, based on a limiting dilution analysis of the number of clonogenic cells produced in a stromal co-culture after 5-8 weeks (Sutherland et al. (1990) Proc. Nat'l Acad. Sci. 87:3584-3588). The LTCIC assay has been shown to correlate with another commonly used stem cell assay, the cobblestone area forming cell (CAFC) assay, and with long-term engrafting potential in vivo (Breems et al, (1994) Leukemia 8:1095). For a review and database of hematopoietic stem cells see Montrone C, Kokkaliaris K D, Loeffler D, Lechner M, Kastenmüller G, Schroeder T, Ruepp A. HSC-explorer: a curated database for hematopoietic stem cells. PLoS One, 2013 Jul. 30; 8(7); e70348. doi: 10.1371/journal.pone.0070348, Print 2013.

The term “hematopoietic stem cells with high reconstitution potential” or “HSCs with high reconstitution potential,” as used herein, refers to animal cells, for example mammalian (including human) cells, that (1) have a greater probability of being able to self-renew than general populations of CD34+ HSCs; (2) have a greater ability to self-renew than general populations of CD34+ HSCs (e.g., able to self-renew faster, more efficiently, in greater numbers, and/or over a longer period of time as compared to a general population of CD34+ HSCs); (3) have a greater probability of being able to differentiate into any of several types of blood cells, including red blood cells and white blood cells, as compared to general populations of CD34+ HSCs; (4) have a greater ability to differentiate into any of several types of blood cells, including red blood cells and white blood cells as compared to general populations of CD34+ HSCs (e.g., able to differentiate into more types of blood cells, able to differentiate in a preferred ratio of types of blood-cells, and/or able to differentiate faster, more efficiently, in greater numbers, and/or over longer periods time as compared to a general population of CD34+ HSCs); (5) have a greater probability of long-term engrafting in vivo than general populations of CD34+ HSCs; (6) have a greater ability for long-term engrafting in vivo than general populations of CD34+ HSCs (e.g., able to engraft more quickly, able to engraft in more types of hosts, able to engraft in more host locations, able to engraft for longer terms, and/or able to engraft with less host rejection and/or complications as compared to a general population of CD34+ HSCs); (7) when engrafted into a NOD/SCID IL2Ry^{null} mice, the mice's CD45+ cells exhibit at least 10% chimerism, as explained in more detail in Example 5 below; (8) when engrafted into primary and secondary NOD/SCID IL2Ry^{null} mice recipients, the cells exhibit only significant engraftment after 12 weeks or more in the primary recipient yet show multilineage reconstitution in the secondary recipient, as explained in more detail in Example 5 below; and/or (9) otherwise outperform general populations of CD34+ HSCs in animal or in vitro stem cell assays, such as assays testing the grafting potential using NOD/SCID IL2Ry^{null} mice. These cells are obtained from animals by the methods disclosed herein, and at least by virtue of their existence in a non-natural environment are different from HSC populations that exist in nature. These differences can include different markers, longer life-spans, different differentiation potential, and/or enrichment relative to the HSC populations that exist in nature.

As used herein, “expansion” includes any increase in cell number. Expansion includes, for example, an increase in the number of hematopoietic stem cells over the number of HSCs present in the cell population used to initiate the culture.

The term “unipotent,” as used herein, refers to cells can produce only one cell type, but have the property of self-renewal, which distinguishes them from non-stem cells.

The term “multipotent” as used herein, is used synonymously with the term “progenitor” and refers to cells which can give rise to any one of several different terminally differentiated cell types. These different cell types are usually closely related (e.g. blood cells such as red blood cells, white blood cells and platelets). For example, mesenchymal stem cells (also known as marrow stromal cells) are multipotent cells, and are capable of forming osteoblasts, chondrocytes, myocytes, adipocytes, neuronal cells, and β-pancreatic islets cells.

The term “pluripotent,” as used herein, refers to cells that give rise to some or many, but not all, of the cell types of an organism. Pluripotent stem cells are able to differentiate into any cell type in the body of a mature organism, although without reprogramming they are unable to de-differentiate into the cells from which they were derived. As will be appreciated, “multipotent” progenitor cells (e.g., neural stem cells) have a more narrow differentiation potential than do pluripotent stem cells.

The term “totipotent,” as used herein, refers to fertilized oocytes, as well as cells produced by the first few divisions of the fertilized egg cell (e.g., embryos at the two and four cell stages of development). Totipotent cells have the ability to differentiate into any type of cell of the particular species. For example, a single totipotent stem cell could give rise to a complete animal, as well as to any of the myriad of cell types found in the particular species (e.g., humans).

The term “CD34” refers to a hematopoietic stem cell antigen selectively expressed on certain hematopoietic stem and progenitor cells derived from human bone marrow, blood, and fetal liver. Yin et al., Blood 90: 5002-5012 (1997); Miaglia, S. et al., Blood 90: 5013-21 (1997). Cells that express CD34 are termed CD34⁺. Stromal cells do not express CD34 and are therefore termed CD34⁻. CD34⁺ cells isolated from human blood may also be capable of differentiating into, for example, cardiomyocytes, endothelial cells, and smooth muscle cells in vivo. See Yeh, et al., Circulation 108: 2070-73 (2003). CD34⁺ cells represent approximately 1% of bone marrow derived nucleated cells; CD34 antigen also is expressed by immature endothelial cell precursors; mature endothelial cells do not express CD34+. Peichev, M. et al., Blood 95: 952-58 (2000). In vitro, CD34+ cells derived from adult bone marrow give rise to a majority of the granulocyte/macrophage progenitor cells (CFU-GM), some colony-forming units-mixed (CFU-Mix) and a minor population of primitive erythroid progenitor cells (burst forming units, erythrocytes or BFU-E). Yeh, et al., Circulation 108: 2070-73 (2003). CD34⁺ cells also may have the potential to differentiate into or to contribute to the development of new myocardial muscle, albeit at low frequency.

Hematopoietic cells can be enriched for CD34⁺ stem cells using anti-CD34 antibodies. For example, techniques have been developed using immunomagnetic bead separation to isolate a highly purified and viable population of CD34⁺ cells from bone narrow mononuclear cells. See, e.g., U.S. Pat. Nos. 5,536.475, 5,035,994, 5,130,144, and 4,965,205.

Growing evidence indicates, however, that not all hematopoietic stem cells express CD34, E.g., Nakauchi, Hirmitsu, Hematopoietic stem cells: Are they CD34-positive or CD34-negative, Nature medicine 4:1009-1010 (1998).

The starting cell population comprising hematopoietic stem cells will be selected by the person skilled in the art depending on the envisaged use. Various sources of cells comprising hematopoietic stem cells have been described in the art, including bone marrow, peripheral blood, neonatal umbilical cord blood, placenta or other sources such as liver, for example, fetal liver.

Further enrichment of HSCs can be accomplished by selecting CD 34+ cells that are also CD38⁻. Such enrichment can be done in accordance with published and/or commercial methodologies for negative selection of cells including, but not limited to, selection based on binding of CD38-specific antibodies to the undesired cell population. Alternative undesired cell populations can be removed from the stem cell pool using antibodies to CD20, CD3, CD14, CD56, CD97 and/or CD235.

The term “CD133” refers to the protein prominin-1, the first in a class of novel pentaspan membrane proteins to be identified in both humans and mice, and was originally classified as a marker of primitive hematopoietic and neural stem cells. Studies have now confirmed the utility of CD133 as a marker of hematopoietic stem cells. Antibodies against CD133 are widely available in the art.

Method of Identification and Isolation of HSCs, for Example, HSCs with High Reconstitution Potential

In one embodiment, the disclosure provides a method for production of an antibody that can be used to identify and isolate human hematopoietic stem cells, for example, HSCs with high reconstitution potential, comprising screening for an antibody that:

binds to 2-3 sialylated lacto-neolacto type structures, from a population of antibodies generated against hematopoietic stem cells; and
can identify and isolate human HSCs, for example, HSCs with high reconstitution potential.

In one embodiment, the hematopoietic stem cells that are used to generate the population of antibodies are CD34+. In one embodiment, the hematopoietic stem cells that are used to generate the population of antibodies are CD34+/CD38−. In one embodiment, the hematopoietic stem cells that are used to generate the population of antibodies are CD34−.

In one embodiment, the 2-3 sialylated lacto-neolacto type structures are chosen from sialyl I (big I, see FIG. 1a), sialyl i (small i, see FIG. 1b), N-acetyl sialyllactoseamine (see FIG. 2a), sialyllacto-N-tetraose (see FIG. 2b), sialyllacto-N-neotetraose (see FIG. 2c), and sialyllactose (see FIG. 3).

In one embodiment, the 2-3 sialylated lacto-neolacto type structure is sialyllactose (see FIG. 3).

In one embodiment, the antibody binds to 2-3 sialylated lacto-neolacto type structures expressed, or present, on the human stem cell marker CD133.

In one embodiment, the antibody binds specifically to 3′SL-CD133.

In one embodiment, the antibody binds specifically to 3′SL-CD133 and not to neuraminidase-treated 3′SL-CD133.

In one embodiment, the antibody can be used to isolate primitive HSCs with high reconstitution potential as functionally determined by in vivo models.

In one embodiment, the in vivo model involves transplanting test cell populations into irradiated mice (e.g., NOD/SCID IL2Ry^{null} mice, also known as NOG mice) and then assessing the long-term repopulating potential of those test cell populations.

In one embodiment, the antibody binds, or has enhanced binding, to CD133 and human-fucosidase-treated-CD133 but does not bind to neuraminidase-treated CD133. In one embodiment, this antibody can be used to isolate primitive HSCs with high reconstitution potential as functionally determined by in vivo models. In one embodiment, the in vivo model involves transplanting test cell populations into irradiated mice (e.g., NOD/SCID IL2Ry^{null} mice, also known as NOG mice) and then assessing the long-term repopulating potential of those test cell populations.

In one embodiment, the hematopoietic stem cells can be isolated from bone marrow. In one embodiment, these cells can be isolated from peripheral blood. In one embodiment, these cells can be isolated from leukopheresis product. In one embodiment, these cells can be isolated from cord blood. In one embodiment, these cells can be isolated from a combination of sources.

In one embodiment, the hematopoietic stem cells can be isolated by FAGS sorting, immunomagnetic beads, and/or affinity matrices.

In one embodiment, the human stem cell marker CD133 is isolated from HSCs. In one embodiment, the human stem cell marker CD133 is isolated from CD34+ HSCs. In one embodiment, the human stem cell marker CD133 is isolated from CD34+/CD38− HSCs.

The present disclosure relates to methods and compositions for the discovery and production of antibodies that can be used to identify and isolate primitive early hematopoietic stem cells with high reconstitution potential. A number of uses for both the cells isolated by such methods and the antibodies generated by the same in regenerative medicine applications are also provided.

In one embodiment, the technologies disclosed herein provide new strategies for the rapid development of diagnostic and therapeutic antibodies for the detection and isolation of hematopoietic stem cells, for example, HSCs with high reconstitution potential. In one embodiment, the technologies disclosed herein provide new strategies for the treatment of hematologic diseases and chronic illnesses, such as leukemias, lymphomas, and myelomas, the treatment of cardiovascular disorders, or the treatment of wounds (e.g., the promotion of wound healing). In one embodiment, the HSCs, cells derived from the HSCs, or antibodies are used for treating advanced follicular lymphoma. In another embodiment, the HSCs, cells derived from the HSCs, or antibodies are used for treatment of a pediatric hematologic disease. In one embodiment, the HSCs, cells derived from the HSCs, or antibodies are used for treating an adult hematologic disease. In another embodiment, the HSCs, cells derived from the HSCs, or antibodies are used to treat acute myeloid leukemia. In one embodiment, the HSCs, cells derived from HSCs, including EPCs derived from the HSCs, media conditioned by the EPCs derived from HSC, or antibodies are used for treating cardiovascular disorders. In one embodiment, the HSCs, EPCs derived from the HSCs, media conditioned by the EPCs derived from HSC, or antibodies are used to treat wounds and/or to promote wound healing. In one embodiment, the HSCs are genetically modified before proliferation and propagation and/or before use in treatment.

In one embodiment, the present disclosure relates to antibodies that can be used to identify and isolate hematopoietic stem cells, for example, HSCs with high reconstitution potential. In one embodiment, the antibodies are specific for 2-3 sialylated lacto-neolacto type structures such as sialyl I (big I, see FIG. 1a), sialyl i (small i, FIG. 1b), N-acetyl sialyllactoseamine (see FIG. 2a), sialyllacto-N-tetraose (see FIG. 2b), sialyllacto-N-neotetraose (see FIG. 2c), and sialyllactose (see FIG. 3).

In one embodiment, a population of cells is provided for the discovery and production of antibodies that can be used to identify and isolate hematopoietic stem cells. In one embodiment, population of cells used to produce antibodies comprises hematopoietic stem cells with high reconstitution potential.

In one embodiment, the disclosure provides antibodies that are specific for 2-3 sialylated lacto-neolacto type structures such as sialyl I (big I, see FIG. 1a), sialyl i (small i, see FIG. 1b), N-acetyl sialyllactoseamine (see FIG. 2a), sialyllacto-N-tetraose (see FIG. 2b), sialyllacto-N-neotetraose (see FIG. 2c), and sialyllactose (see FIG. 3), the antibodies being produced by a method comprising injecting mice with CD34⁺, CD38⁻ HSCs and screening the resultant antibodies for those that bind to 2-3 sialylated lacto-neolacto type structures such as sialyl I (big I), sialyl i (small i), and sialyllactose coated on multiwell plates.

The monoclonal antibodies (MAbs) of the disclosure can be produced by a variety of techniques, including conventional monoclonal antibody methodology, e.g., the standard somatic cell hybridization technique of Kohler and Milstein, 1975, Nature 256:495. Somatic cell hybridization procedures may be used or other techniques for producing monoclonal antibodies can be employed, including, e.g., viral or oncogenic transformation of B-lymphocytes.

One skilled in the art can engineer mouse strains deficient in mouse antibody production with large fragments of the human Ig loci so that such mice produce human antibodies in the absence of mouse antibodies. Large human Ig fragments may preserve the large variable gene diversity as well as the proper regulation of antibody production and expression. By exploiting the mouse machinery for antibody diversification and selection and the lack of immunological tolerance to human proteins, the reproduced human antibody repertoire in these mouse strains yields high affinity antibodies against any antigen of interest, including human antigens. Using the hybridoma technology, antigen-specific human MAbs with the desired specificity may be produced and selected.

In alternative embodiments, antibodies of the disclosure can be expressed in cell lines other than hybridoma cell lines. In these embodiments, sequences encoding particular antibodies can be used for transformation of a suitable mammalian host cell. According to these embodiments, transformation can be achieved using any known method for introducing polynucleotides into a host cell, including, for example packaging the polynucleotide in a virus (or into a viral vector) and transducing a host cell with the virus (or vector) or by transfection procedures known in the art. Such procedures are exemplified by U.S. Pat. Nos. 4,399,216, 4,912,040, 4,740,461, and 4,959,455. Generally, the transformation procedure used may depend upon the host to be transformed. Methods for introducing heterologous polynucleotides into mammalian cells are well known in the art and include, but are not limited to, dextran-mediated transfection, calcium phosphate precipitation, polybrene mediated transfection, protoplast fusion, electroporation, encapsulation of the polynucleotide(s) in liposomes, and direct microinjection of the DNA into nuclei.

In one embodiment, the disclosure provides for antibodies capable of binding to 3′-sialylated lactose. In one embodiment, the antibodies are capable of binding to 3′-sialylated lactose on the surface of human hematopoietic stem cells.

In another embodiment, the disclosure provides for antibodies capable of binding specifically to 2-3 sialyated lacto-neolacto type structures expressed, or present, on the human stem cell marker CD133. In one embodiment, this CD133 is isolated from HSCs. In one embodiment, CD133 is isolated from hematopoietic stem cells with high reconstitution potential

Screening for hybridomas/antibodies that are capable of binding specifically to 2-3 sialyated lacto-neolacto type structures expressed on the human stem cell marker 00133 can be achieved by any of a plurality of techniques available to one of ordinary skill in the art. In one embodiment, the screening method comprises purifying CD133 molecules extracted from human hematopoietic stem and progenitor cells by affinity chromatography using anti-CD133 antibodies immobilized on an affinity matrix. In another embodiment, specific glycoforms of CD133 that express 2-3 sialylated lacto structures are then further purified from these CD133 molecules by lectin affinity chromatography using MAA lectin (Maakia amenuris) immobilized on an affinity matrix. MAA lectin binds to 2-3 sialylated lacto structures.

In one embodiment, a purified specific glycoform of CD133 (e.g., 3′sialylated lacto/neolacto structures-CD133, or 3′SL-CD133), is coated in wells of a microtiter plate. In one embodiment, half of the 3′SL-CD133-coated wells are treated with neuraminidase. Neuraminidase cleaves terminal sialic acid residues on the epitope that binds the desirable antibody being screened for. Those antibodies that specifically bind to 3′SL-CD133 and not to neuraminidase-treated 3′SL-CD133 are then purified and/or cloned.

In one embodiment, the antibodies that specifically bind to 3′SL-CD133 and not to neuraminidase-treated 3′SL-CD133 are then tested for their ability to isolate HSCs with high reconstitution potential as functionally determined by in vivo models.

In another embodiment, purified CD133 is coated in wells of a microtiter plate. Half of the CD133-coated wells are treated with human fucosidase. Fucosidase removes the Lewis fucose that is on the undesirable, more differentiated carbohydrate structure on CD133 (sialyl Lex). Hybridomas are then selected for their ability to bind, or have enhanced binding, to CD133 and human-fucosidase-treated-CD133 but not to neuraminidase-treated CD133. In one embodiment, these antibodies can then be tested for their ability to isolate HSCs with high reconstitution potential as functionally determined by in vivo models.

In one embodiment, the present disclosure relates to human hematopoietic stem cells, for example, HSCs with high reconstitution potential. The disclosure provides a method of isolating such cells for potential use in therapy.

In another embodiment, a composition is provided comprising a stem cell produced according to one of the methods of the disclosure.

In another embodiment, the stem cells produced according to one of the methods of the disclosure may be used directly in therapeutic strategies for treating a variety of disease states and conditions, including hematologic diseases, disorders or conditions (e.g., thalassemias, sickle cell disease, leukemias, lymphomas, myelomas).

In another embodiment, the stem cells produced according to one of the methods of the disclosure may be genetically modified before use in gene therapy strategies for treating a variety of disease states and conditions, including genetic diseases, disorders or conditions such as genetic hematologic diseases, disorders or conditions (e.g., sickle cell, thalassemia, or severe combined immune deficiency).

In another embodiment, the stem cells produced according to one of the methods of the disclosure may be partially or full differentiated into cells of a desired lineage and those cells may be used in therapeutic strategies for treating a variety of disease states and conditions, including hematologic diseases, disorders conditions (e.g., thalassemias, sickle cell disease, leukemias, lymphomas, myelomas), cardiovascular disorders, or to treat wounds (e.g., to promote wound healing). In one embodiment, the cells or antibodies are used for treating advanced follicular lymphoma. In another embodiment, the cells or antibodies are used for treatment of a pediatric hematologic disease. In one embodiment, the cells or antibodies are used for treating an adult hematologic disease. In another embodiment, the cells or antibodies are used to treat acute myeloid leukemia.

In another embodiment, the genetically modified stem cells produced according to one of the methods of the disclosure may be partially or full differentiated into genetically modified cells of a desired lineage and those genetically modified cells may be used in therapeutic strategies for treating a variety of disease states and conditions, such as genetic hematologic diseases, disorders or conditions (e.g., sickle cell, thalassemia, or severe combined immune deficiency).

In one embodiment, the primitive early HSCs, for example, HSCs with high reconstitution potential are used for treatment of cardiovascular disorders or treatment of wounds (e.g., to promote wound healing). In one embodiment, EPCs may be propagated from the HSCs for use in the treatment of cardiovascular disorders or the treatment of wounds (e.g., to promote wound healing), or to condition media for use in the treatment of cardiovascular disorders or the treatment of wounds (e.g., to promote wound healing).

Culture media suitable for the ex vivo culturing of HSCs, including the culturing of HSCs that have been genetically modified, according to the practice described herein are well known in the art, e.g. as disclosed in U.S. Pat. No. 6,030,836, and by J. Hartshorn, et al., “Ex Vivo Expansion of Hematopoietic Stem Cells Using Defined Culture Media” in Cell Technology for Cell Products, Chapter III, pages 221-224. Such culture media include but are not limited to high glucose Dulbecco's Modified Eagles Medium (DMEM) with L-Glutamine which is well known and readily commercially available. The media can be supplemented with recombinant human basic fibroblast growth factor (rhbFGF) and contain sera, such as human serum, and antibiotics. Cell cultures are maintained in a CO₂ atmosphere, e.g., 5% to 12%, to maintain pH of the culture fluid, and incubated at 37° C. in a humid atmosphere. Suitable chemically defined serum-free media are described in U.S. Ser. No. 08/464,599 and WO96/39487, and “complete media” are described in U.S. Pat. No. 5,486,359 and these are hereby incorporated by reference. Chemically defined medium comprises a minimum essential medium such as Iscove's Modified Dulbecco's Medium (IMDM) (Gibco), supplemented with human serum albumin, human Ex Cyte lipoprotein, transferrin, insulin, vitamins, essential and non-essential amino acids, sodium pyruvate, glutamine and a mitogen. These media stimulate cell growth without differentiation. As used herein, a mitogen refers to an agent that stimulates cell division of a cell. Such an agent can be a chemical, usually some form of a protein that encourages a cell to commence cell division triggering mitosis. Other examples of culture medium include RPMI 1640, Iscove's modified Dulbecco's media (IMDM), and Opti-MEM SFM (Invitrogen Inc.). Chemically Defined Medium comprises a minimum essential medium such as Iscove's Modified Dulbecco's Medium (IMDM) (Gibco), supplemented with human serum albumin, human Ex Cyte lipoprotein, transferrin, insulin, vitamins, essential and non-essential amino acids, sodium pyruvate, glutamine and a mitogen is also suitable. HSCs can also be expanded according to the methods described in Example 6.

The route of administration of the antibodies and cells of the present disclosure (whether the pure antibody/cell, a labeled antibody/cell, an antibody fused to a toxin, etc.) is in accord with known methods.

In certain embodiments, the pharmaceutical composition may contain formulation materials for modifying, maintaining or preserving, for example, the pH, osmolarity, viscosity, clarity, color, isotonicity, odor, sterility, stability, rate of dissolution or release, adsorption or penetration of the composition. In such embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin; gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, trimethamine, lecithin, cholesterol, tyloxapol); stability enhancing agents (such as sucrose or sorbitol); tonicity enhancing agents (such as alkali metal halides, for example, sodium or potassium chloride, mannitol sorbitol); delivery vehicles; diluents; excipients and/or pharmaceutical adjuvants. See, Remington's Pharmaceutical Sciences, 18th Edition, (A. R. Gennaro. ed.), 1990, Mack Publishing Company.

In certain embodiments, a suitable pharmaceutical composition comprising the antibodies and/or cells described herein will be determined by one skilled in the art depending upon, for example, the intended route of administration, delivery format and desired dosage. See, for example, Remington's Pharmaceutical Sciences, supra. In certain embodiments, such compositions may influence the physical state, stability, rate of in vivo release and rate of in vivo clearance of the antibodies of the disclosure.

In certain embodiments, the primary vehicle or carrier in a pharmaceutical composition may be either aqueous or non-aqueous in nature. For example, a suitable vehicle or carrier may be water for injection, physiological saline solution or artificial cerebrospinal fluid, possibly supplemented with other materials common in compositions for parenteral administration. Neutral buffered saline or saline mixed with serum albumin are further exemplary vehicles. In some embodiments, pharmaceutical compositions comprise Tris buffer of about pH 7.0-8.5, or acetate buffer of about pH 4.0-5.5, and may further include sorbitol or a suitable substitute therefor. In certain embodiments of the disclosure, the compositions may be prepared for storage by mixing the selected composition having the desired degree of purity with optional formulation agents (Remington's Pharmaceutical Sciences, supra) in the form of a lyophilized cake or an aqueous solution. Further, in certain embodiments, the product may be formulated as a lyophilizate using appropriate excipients such as sucrose.

The pharmaceutical compositions of the disclosure can be selected for parenteral delivery. The compositions may be selected for inhalation or for delivery through the digestive tract, such as orally. Preparation of such pharmaceutically acceptable compositions is within the skill of the art.

The formulation components may be present in concentrations that are acceptable to the site of administration. In certain embodiments, buffers are used to maintain the composition at physiological pH or at a slightly lower pH, typically within a pH range of from about 5 to about 8.

When parenteral administration is contemplated, the therapeutic compositions for use in this disclosure may be provided in the form of a pyrogen-free, parenterally acceptable aqueous solution comprising the desired antibody and/or cells in a pharmaceutically acceptable vehicle. An example of a suitable vehicle for parenteral injection is sterile distilled water in which the antibody is formulated as a sterile, isotonic solution, properly preserved. In certain embodiments, the preparation can involve the formulation of the desired molecule with an agent, such as injectable microspheres, bio-erodible particles, polymeric compounds (such as polylactic acid or polyglycolic acid), beads or liposomes, that may provide controlled or sustained release of the product which can be delivered via depot injection. In certain embodiments, hyaluronic acid may also be used, having the effect of promoting sustained duration in the circulation. In certain embodiments, implantable delivery devices may be used to introduce the desired antibody and/or cells.

Pharmaceutical compositions of the disclosure can be formulated for inhalation. In these embodiments, the antibodies are formulated as a dry powder for inhalation. In some embodiments, antibody inhalation solutions may also be formulated with a propellant for aerosol delivery. In certain embodiments, solutions may be nebulized. Pulmonary administration and formulation methods therefore are further described in International Patent Publication No, WO94/20069, incorporated by reference, which describes pulmonary delivery of chemically modified proteins.

It is also contemplated that formulations can be administered orally. Antibodies that are administered in this fashion can be formulated with or without carriers customarily used in the compounding of solid dosage forms such as tablets and capsules. In certain embodiments, a capsule may be designed to release the active portion of the formulation at the point in the gastrointestinal tract when bioavailability is maximized and pre-systemic degradation is minimized. Additional agents can be included to facilitate absorption of the antibody. Diluents, flavorings, low melting point waxes, vegetable oils, lubricants, suspending agents, tablet disintegrating agents, and binders may also be employed.

Some pharmaceutical compositions of the disclosure comprise an effective quantity of one or a plurality of the antibodies herein described in a mixture with non-toxic excipients that are suitable for the manufacture of tablets. By dissolving the tablets in sterile water, or another appropriate vehicle, solutions may be prepared in unit-dose form. Suitable excipients include, but are not limited to, inert diluents, such as calcium carbonate, sodium carbonate or bicarbonate, lactose, or calcium phosphate; or binding agents, such as starch; gelatin, or acacia; or lubricating agents such as magnesium stearate, stearic acid, or talc.

Additional pharmaceutical compositions will be evident to those skilled in the art, including formulations involving sustained- or controlled-delivery formulations. Techniques for formulating a variety of other sustained- or controlled-delivery means, such as liposome carriers, bio-erodible microparticles or porous beads and depot injections, are also known to those skilled in the art. See for example; International Patent Publication No, WO93/15722, which describes controlled release of porous polymeric microparticles for delivery of pharmaceutical compositions. Sustained-release preparations may include semipermeable polymer matrices in the form of shaped articles, e.g., films or microcapsules. Sustained release matrices may include polyesters, hydrogels, polylactides (as disclosed in U.S. Pat. No. 3,773,919 and European Patent Application Publication No. EP 058481), copolymers of L-glutamic acid and gamma ethyl-L-glutamate (Sidman et al., 1983, Biopolymers 22:547-556), poly (2-hydroxyethyl-methacrylate) (Langer et al., 1981, J. Biomed. Mater. Res. 15:167-277 and Langer, 1982, Chem. Tech. 12:98-105), ethylene vinyl acetate (Langer et al., supra) or poly-D(−)-3-hydroxybutyric acid (European Patent Application Publication No. EP 133,988). Sustained release compositions may also include liposomes that can be prepared by any of several methods known in the art. See, e.g., Eppstein et al., 1985, Proc. Natl. Acad. Sci. USA 82:3688-3692; European Patent Application Publication Nos. EP 036,676, EP 088,046, and EP 143,949.

Pharmaceutical compositions used for in vivo administration are typically provided as sterile preparations. Sterilization can be accomplished by filtration through sterile filtration membranes. When the composition is lyophilized, sterilization using this method may be conducted either prior to or following lyophilization and reconstitution. Compositions for parenteral administration can be stored in lyophilized form or in a solution. Parenteral compositions generally are placed into a container having a sterile access port, for example, an intravenous solution bag or vial having a stopper pierceable by a hypodermic injection needle.

Once the pharmaceutical composition has been formulated, it may be stored in sterile vials as a solution, suspension, gel, emulsion, solid, or as a dehydrated or lyophilized powder. Such formulations may be stored either in a ready-to-use form or in a form (e.g., lyophilized) that is reconstituted prior to administration. Pharmaceutical compositions suitable for use include compositions wherein one or more of the present antibodies and/or cells are contained in an amount effective to achieve their intended purpose. More specifically, a therapeutically effective amount means an amount of antibody and/or cells effective to prevent, alleviate or ameliorate symptoms of disease or prolong the survival of the subject being treated. Determination of a therapeutically effective amount is well within the capability of those skilled in the art, especially in light of the detailed disclosure provided herein. Therapeutically effective dosages may be determined by using in vitro and in vivo methods.

In one embodiment, the present antibodies also may be utilized to detect HSCs in vivo or ex vivo. Detection in vivo is achieved by labeling the antibodies described herein, administering the labeled antibody to a subject, and then imaging the subject. Examples of labels useful for diagnostic imaging in accordance with the present disclosure are radiolabels such as I¹²³, I¹³¹, I¹¹¹, Tc^{99m}, P³², I¹²⁵, H³, C¹⁴, and Rh¹⁸⁸, fluorescent labels such as fluorescein and rhodamine, nuclear magnetic resonance active labels, positron emitting isotopes detectable by a positron emission tomography (“PET”) scanner, chemiluminescers such as luciferin, and enzymatic markers such as peroxidase or phosphatase. Short-range radiation emitters, such as isotopes detectable by short-range detector probes, such as a transrectal probe, can also be employed. The antibody can be labeled with such reagents using techniques known in the art. For example, see Wensel and Meares, Radioimmunoimaging and Radioimmunotherapy, Elsevier, N.Y. (1983), which is hereby incorporated by reference, for techniques relating to the radiolabeling of antibodies. See also D. Colcher et al., “Use of Monoclonal Antibodies as Radiopharmaceuticals for the Localization of Human Carcinoma Xenografts in Athymic Mice”, Meth. Enzymol, 121: 802-816 (1986), which is hereby incorporated by reference.

A radiolabeled antibody in accordance with this disclosure can be used for in vitro diagnostic tests. The specific activity of an antibody, binding portion thereof, probe, or ligand, depends upon the half-life, the isotopic purity of the radioactive label, and how the label is incorporated into the biological agent. In immunoassay tests, the higher the specific activity, in general, the better the sensitivity. Procedures for labeling antibodies with the radioactive isotopes are generally known in the art.

The radiolabeled antibody can be administered to a patient where it is localized to HSCs bearing the antigen with which the antibody reacts, and is detected or “imaged” in vivo using known techniques such as radionuclear scanning using e.g., a gamma camera or emission tomography. See, e.g., A. R. Bradwell et al., “Developments in Antibody Imaging,” Monoclonal Antibodies for Cancer Detection and Therapy, R. W. Baldwin et al. (eds.), pp. 65-85 (Academic Press 1985), which is hereby incorporated by reference. Alternatively, a positron emission transaxial tomography scanner, such as designated Pet VI located at Brookhaven National Laboratory, can be used where the radiolabel emits positrons (e.g., C¹¹, F¹⁸, O¹⁵, and N¹³).

Fluorophore and chromophore labeled biological agents can be prepared from standard moieties known in the art. Since antibodies and other proteins absorb light having wavelengths up to about 310 nm, the fluorescent moieties should be selected to have substantial absorption at wavelengths above 310 nm, for example, above 400 nm. A variety of suitable fluorescers and chromophores are described by Stryer, Science, 162:526 (1968) and Brand, L. et al., Annual Review of Biochemistry, 41:843-868 (1972), which are hereby incorporated by reference. The antibodies can be labeled with fluorescent chromophore groups by conventional procedures such as those disclosed in U.S. Pat. Nos. 3,940,475, 4,289,747, and 4,376,110, which are hereby incorporated by reference.

In another embodiment in accordance with the present disclosure, methods are provided for treatment, monitoring the progress, and/or effectiveness of a therapeutic treatment.

In one embodiment of each of the therapeutic methods described herein, the subject is first diagnosed with a need for increased or replacement HSCs. In one embodiment of each of the therapeutic methods described herein, the subject has a need for increased blood cells. In another embodiment, the subject has a disease chosen from hematologic diseases, disorders or conditions (e.g., thalassemias, sickle cell disease, leukemias, lymphomas, myelomas, etc). In one embodiment, the cells or antibodies are used for treating advanced follicular lymphoma. In another embodiment, the cells or antibodies are used for treatment of a pediatric hematologic disease. In one embodiment, the cells or antibodies are used for treating an adult hematologic disease. In another embodiment, the cells or antibodies are used to treat acute myeloid leukemia.

In one embodiment of each of the therapeutic methods described herein, the subject is first diagnosed with a cardiovascular disorder and/or a need for HSCs or EPCs, and the cells or antibodies are used to treat the cardiovascular disorder. In another embodiment the subject is suffering from a wound, and the cells or antibodies are used to treat the wound (e.g., to promote wound healing. In another embodiment, cells propagated from the HSCs are used to condition media that is used to treat the cardiovascular disorder or treat wounds (e.g., to promote wound healing).

In one embodiment of each of the therapeutic methods described herein, the subject is first diagnosed with a genetic disease, disorder or condition and/or a need for genetically modified HSCs. In one embodiment, the genetically modified HSCs are used for treating diseases or disorders of the blood such as sickle cell, thalassemia, and severe combined immune deficiency.

In one embodiment, described herein is a method for transplanting a population of human HSCs with high reconstitution potential. In one embodiment, described herein is a method for transplanting a population of cells derived from human HSCs with high reconstitution potential. In one embodiment, described herein is a method for transplanting a population of genetically modified human hematopoietic stem cells with high reconstitution potential.

Certain methods disclosed herein are applicable to any situations wherein a greater percentage or number of HSCs is desired, in clinical research, for drug discovery, or for engraftment in human hematopoietic stem cell transplantation, for example, to rescue patients after cytoablative therapies. For example, in bone marrow transplants, it is known that the higher the number or percentage of HSCs implanted into a recipient, the greater percentage of engraftment of the donor HSCs in the recipient.

Certain methods disclosed herein are applicable to any situations wherein genetically modified HSCs is desired, in clinical research, for drug discovery, or for engraftment in human hematopoietic stem cell transplantation, for example, for gene therapies.

In one embodiment, described herein is a pharmaceutical composition comprising an isolated cell population comprising an HSC, for example, an HSC with high reconstitution potential, and a pharmaceutically-acceptable carrier.

In one embodiment, described herein is a pharmaceutical composition comprising an isolated cell population comprising cells propagated from an HSC, for example, an HSC with high reconstitution potential, and a pharmaceutically-acceptable carrier.

In one embodiment, described herein is a pharmaceutical composition comprising an isolated cell population comprising a genetically modified hematopoietic stem cell, for example, an HSC with high reconstitution potential, and a pharmaceutically-acceptable carrier.

Depending on the specific embodiment, pharmaceutical compositions described herein can include, for example, agents that stimulate or promote HSC expansion/self-renewal/long-term culture initiating colony formation capability, or cells generated from such expansions. Accordingly, formulations for administration of such compositions will depend upon specific embodiments. Agents that promote expansion, for example, can be administered by any suitable route for that agent. Routes of administration include, but are not limited to, intradermal, intramuscular, intraperitoneal, intravenous, and subcutaneous routes. Routes of administration may also include direct administration, e.g., to the site of a wound.

While there are methods for increasing mobilization of HSCs into circulation from the bone marrow, the methods disclosed herein can be used in conjunction with the mobilization methods to increase the amount of circulating HSCs and the HSCs in the bone marrow of the donor prior to harvesting. In addition, the methods disclosed herein can be used to increase the amount of HSCs after the cells are harvested from a donor but before to cells are transplanted into the recipient. The HSCs harvested from a donor are initially cultured ex vivo and expanded in culture by the methods disclosed herein. When the number of HSCs has reached a desired amount, the cultured HSCs can be harvested and implanted into the recipient.

In one embodiment, the HSCs are isolated from a subject, optionally cultured to expand in numbers, harvested, and transplanted back into the same subject, i.e., an autologous cell transplant. In one embodiment, the HSCs are isolated from a subject, genetically modified, and optionally cultured to expand in numbers, harvested, and transplanted back into the same subject, i.e., an autologous cell transplant.

In another embodiment, the HSCs are isolated from a donor who is an HLA-type match with a recipient subject wherein the donor and recipient are two separate individuals. This is allogeneic transplantation. Donor-recipient antigen type-matching is well known in the art. The HLA-types include HLA-A, HLA-B, HLA-C, and HLA-D. Typically, these represent the minimum number of cell surface antigen matching required for transplantation.

In one embodiment, the isolated cell population comprising an HSC or a genetically modified HSC is cryopreserved before being transplanted.

The transplantation method is not limited by the nature of the donor or recipient. In some embodiments, the donor and recipient are both human. The transplant recipient can be fully- or partially-allogeneic to the donor. The transplantation can be autologous. Transplant recipients or donors can be less than five years of age, from 1 to 10 years of age, from 5 to 15 years of age, from 10 to 20 years of age, from 15 to 25 years of age, from 20 to 30 years of age, from 25 to 35 years of age, from 30 to 40 years of age, from 35 to 45 years of age, from 40 to 50 years of age, from 45 to 55 years of age, from 50 to 60 years of age, from 55 to 65 years of age, from 60 to 70 years of age, or 70 years of age or older.

In another embodiment, the subject being treated has received radiation (e.g., has been irradiated) at a sub-lethal or lethal dose as an adjunct to transplantation.

Other embodiments of the disclosure will be apparent to those skilled in the art from consideration of the specification and practice of the disclosure disclosed herein.

HSCs, for example, HSCs with high reconstitution potential, have numerous uses in the clinic. For a review see, for example, Weissman I L, Shizuru J A, The origins of the identification and isolation of hematopoietic stem cells, and their capability to induce donor-specific transplantation tolerance and treat autoimmune diseases, Blood, 2008 Nov. 1, 112(9):3543-53.

Numerous specific examples of the use of stem cell transplantation in human therapies can be found in the art. See, for example, Hematopoietic Stem Cell Transplantation, edited by Anthony D. Ho, Rainer Haas, and Richard E. Champlin, Marcel Dekker Inc. 2000; Schrauder A, von Stackelberg A, Schrappe M, Cornish J, Peters C, ALL-BFM Study Group, EBMT PD WP, I-BFM Study Group, Allogeneic hematopoietic SCT in children with ALL: current concepts of ongoing prospective SCT trials, Bone Marrow Transplant, 2008 June, 41 Suppl 2:S71-4; Copelan E A, Hematopoietic stem-cell transplantation, N Engl J Med, 2006 Apr. 27, 354(17):1813-26; Kim S W, Hematopoietic stem cell transplantation for follicular lymphoma: optimal timing and indication, J Clin Exp Hematop, 2014, 54(1):39-47; Choi S W, Reddy P, Current and emerging strategies for the prevention of graft-versus-host disease, Nat Rev Clin Oncol, 2014 September, Vol. 11, pp. 536-47; Rambaldi A, Biagi E, Bonini C, Biondi A, Introna M, Cell based strategies to manage leukemia relapse: efficacy and feasibility of immunotherapy approaches, Leukemia, 29:1-10, 2014 Jul. 8, doi: 10.1038/leu.2014.189. [Epub ahead of print]; Kekre N, Antin J H, Hematopoietic stem cell transplantation donor sources in the 21st century: choosing the ideal donor when a perfect match doesn't exist, Blood, 2014 Jul. 17, Vol. 124(3), pp. 334-343; and Tsirigotis P, Shimoni A, Nagler A, The expanding horizon of immunotherapy in the treatment of malignant disorders: Allogeneic hematopoietic stem cell transplantation and beyond, Ann Med, 2014, Vol. 46(6), pp. 384-396.

The route of administration, the number of transplanted cells per body weight, the pre-transplantation and post-transplantation treatment of the recipient, and the rate and frequency of administration of the HSCs or HSCs with high reconstitution potential can be determined by one of ordinary skill in the art using routine methods. In one embodiment, the method of administration is intravenous infusion. The number of cells transfused/transplanted will take into consideration factors such as sex, age, weight, the types of disease or disorder, stage of the disease or disorder, the percentage of the desired cells in the cell population (e.g., purity of cell population), and the cell number needed to produce a therapeutic benefit. A variety of adjunctive treatments may be used with the methods described herein. In one aspect, the adjunctive treatments include, among others, anti-fungal agents, anti-bacterial agents, and anti-viral agents.

As mentioned above, the amount of the cells needed for achieving a therapeutic effect will be determined empirically in accordance with conventional procedures for the particular purpose. Generally, for administering cells for therapeutic purposes, the cells are given at a pharmacologically effective dose. By “pharmacologically effective amount” or “pharmacologically effective dose” is an amount sufficient to produce the desired physiological effect or amount capable of achieving the desired result, for example, for engraftment or survival of a subject. Therapeutic benefit also includes halting or slowing the progression of the underlying disease or disorder, regardless of whether improvement is realized.

### EXAMPLES

### Example 1: Isolation of Human CD34⁺, CD38⁻ HSCs for Immunization

Human CD34+ hematopoietic stem cells harvested using positive immunomagnetic selection can be obtained from Lonza or any other commercial sources. They can be derived from, for example, bone marrow, peripheral blood, leukopheresis product, and/or cord blood. In one example, low-density bone marrow mononuclear cells (less than 1.077 g/mL) are separated over Ficoll-Hypaque. CD34+ cells are enriched using a commercially available cell separation system kit from Cell Pro Inc (Bothel, Wash.), washed twice with 1% BSA in phosphate-buffered saline (PBS) and resuspended in 1% BSA to a concentration of 2×10⁸ cells/mL and incubated for 25 minutes with a biotinylated anti-CD34 IgM monoclonal antibody (MoAb) (12.8) at room temperature. The cells are washed with 1% BSA to remove unbound antibody, then resuspended at 2×10⁷, cell/mL in 5% BSA and loaded onto an avidin column. The adsorbed CD34+ cells are released by manually squeezing the gel bed, resuspended in IMDM with 20% FCS, and counted on a Coulter counter (Coulter Electronics, Hialeah, Fla.).

Alternatively, mononuclear cells are isolated from human bone marrow using Ficoll Hypaque (Pharmacia, Piscataway, N.J.) density centrifugation. The mononuclear fraction is then pre-enriched for CD34+ cells using the mini-Magnetic Activated Cell Sorter system (Miltenyi Biotec, Auburn, Calif.), which provides an 85% to 90% pure CD34+ population. The resultant cells are then incubated with fluorescein isothiocyanate (FITC)-labeled anti-CD34 (HPCA2-FITC; Becton Dickinson, San Jose, Calif.) and phycoerythrin (PE)-labeled anti-CD38 (Leu 17-PE; Becton Dickinson), and CD34+/CD38− cells are isolated by FACSVantage (Becton Dickinson) to a purity of more than 99%. CD34+CD38− cells are acquired as those with high CD34 antigen expression and CD38 fluorescence less than half of the maximum PE fluorescence of the isotype control.

### Example 2: Isolation of CD133 for Generation of Antibodies Against 2-3 Sialylated Lacto-Neolacto Type Structures Such as Sialyl I (Big I), Sialyl i (Small i and Sialyl Lactose on the Surface of CD133

Isolation and purification of CD133 from human hematopoietic stem and progenitor cells can be done by any of several methods known to one of ordinary skill in the art. In one example, CD133+-cells (isolated using, for example, anti-CD133-coated magnetic beads/Diamond CD133 isolation kit from Miltenyi Biotec, Auburn, Calif.) (2×10⁹) are washed with PBS and lysed in extraction buffer. The cells are vortexed intermittently for 5 minutes at room temperature and then left on ice for 20 minutes. Cell nuclei and debris are removed by centrifugation at 10,000 g for 10 minutes at 4° C. The lysate/supernatant is filtered through a 0.2-μm filter before loading onto 0.5 mL of an anti-CD133 affinity column equilibrated in wash buffer (0.125 M NaCl, 25 mM Tris, pH 8.0, 0.01% NaN₃, 2.5 mM EDTA, and 0.1% Brij). The column is then washed extensively with wash buffer, and the CD133 antigen is eluted in 50 mM ethanolamine, pH 11.5, 0.1% Brij, and 0.01% NaN₃. The pH is adjusted to neutral with HCl. Removal of left-over contaminating proteins can be done by additional chromatography including passage of the antigen eluate over a 300 μl bed volume DEAE column equilibrated in wash buffer and a second affinity-chromatography step. The purity and identity of the CD133 antigen eluted can be confirmed by, for example, SDS-PAGE and Western blot analysis.

### Example 3: Generation of Antibodies Against 2-3 Sialylated Lacto-Neolacto Type Structures Such as Sialyl I (Big I), Sialyl i (Small i), and Sialyllactose

In one example, to generate murine monoclonal antibodies (Mabs) against 2-3 sialylated lacto-neolacto type structures, such as sialyl I (big I), sialyl i (small i), and sialyllactose, BALB/c mice are immunized at least 3-times s.c. (e.g., footpad), twice weekly, with 5×10⁵ CD34⁺, CD38⁻ HSCs in 0.03 mL PBS, pH7.4 The first immunization is done in presence of Complete Freund Adjuvant (Sigma, St Louis, Md., USA). Incomplete Freund adjuvant (Sigma) is added to the subsequent immunizations. In addition, the cells can be incubated with 1:100 phytohemagglutinin (PHA) for 10 minutes before injection. Three days prior to the fusion, immunized mice are boosted with 5×10⁵ CD34⁺, CD38⁻ HSC. On approximately day 21, splenocytes and lymphocytes are prepared from the immunized mice by perfusion of the spleen and by mincing of the proximal lymph nodes, respectively, harvested and fused to SP2/0-Ag14 myeloma cells (ATCC, Rockville, Md., USA). The fusion protocol can be as described by Kohler and Milstein (Nature, 256:495-497, 1975).

Fused cells are then subjected to HAT selection. In general, for the preparation of monoclonal antibodies or their functional fragments, especially of murine origin, it is possible to refer to techniques which are described, for example, in the manual “Antibodies” (Harlow and Lane, Antibodies: A Laboratory Manual, Cold Spring Harbor Laboratory, Cold Spring Harbor N.Y., pp. 726, 1988). Approximately 10 days after the fusion, colonies of hybrid cells are screened. For the primary screen, supernatants of hybridomas are evaluated for the secretion of Mabs raised against one of the 2-3 sialylated lacto-neolacto type structures such as sialyl I (big I), sialyl (small I), and sialyllactose by ELISA. In one example, the supernatants are evaluated for those that bind to 3′sialylated lactose conjugated to human serum albumin coated (HSA) on ELISA plates but do not bind to HAS alone. Positive reactors on this test are amplified, cloned and a set of hybridomas is recovered, purified and screened for its ability to specifically bind to 2-3 sialylated lacto-neolacto type structures expressed on the human stem cell marker CD133. Isotype controls are used in each experiment (Sigma, ref M90351MG).

### Example 4: Selection of Hybridomas Capable of Specifically Binding to 2-3 Sialylated Lacto-Neolacto Type Structures Expressed on the Human Stem Cell Marker CD133

Each well of a 96-well plate is coated with CD133 (extracted from human hematopoietic stem and progenitor cells) in PBS (e.g., 1 μg/mL, 50 μL) and incubated for 1 hour at 37° C. Hybridoma culture supernatants containing the antibodies to be selected are then added to separate wells and incubated for an additional 1 hour at 37° C. The wells are then washed with PBS-Tween and incubated with labeled secondary anti-mouse antibodies (e.g., HRP-conjugated goat anti-mouse antibodies of various isotypes) for 1 hour at 37° C. Following this incubation, the wells are washed with PBS and bound antibodies visualized according to the secondary antibody label (e.g., using a colorimetric assay with o-phenylene diamine as a chromogenic substrate for HRP; absorbance read using a microplate reader at 492 nm).

Specific glycoforms of CD133 that express 2-3 sialylated lacto structures can be purified by lectin affinity chromatography using Maakia amurensis (MAA) lectin. MAA lectin chromatography gels can be obtained from commercial sources (e.g., EYlabs). Gels are poured into small columns (e.g., plastic mini-columns) and washed with 10 times the gel volume of buffer. CD133 extracted from human hematopoietic stem and progenitor cells is applied to the column and the unbound material washed from the column with buffer. The bound material is then eluted with the appropriate carbohydrate, such as 2-3 sialyllacto-neolacto-type structures in the buffer of choice.

Some of the desired antibodies bind to 3′sialylated-CD133 (3)SL-CD133) but do not bind to neuraminidase-treated 3′SL-CD133. Such antibodies can be tested for isolation of primitive HSCs with high reconstitution potential using any of the well-established functional in vivo models.

In other cases, the desired antibodies bind to CD133 (coated wells) and to human fucosidase-treated CD133 (coated wells) but do not bind to neuraminidase-treated CD133. Such antibodies can be tested for isolation of primitive HSCs with high reconstitution potential using any of the well-established functional in vivo models.

### Example 5: Screening of Hybridomas/Antibodies Capable of Isolating HSCs with High Reconstitution Potential

There are several methods known to one of ordinary skill in the art that serve as tools to assay the self-renewal ability and reconstitution potential of isolated hematopoietic cell populations. One of such methods involves transplanting test cell populations into irradiated mice (e.g., NOD/SCID IL2Ry^{null} mice, also known as NOG mice) and then assessing the long-term repopulating potential of those test cell populations. See, for example, Majeti R, Park C Y, and Weissman I L, (2007) Identification of a Hierarchy of Multipotent Hematopoietic Progenitors in Human Cord Blood, Cell Stem Cell, Vol. 1 (6):635-645 and Wang, J. C., Lapidot, T., Cashman, J. D., Doedens, M., Addy, L, Sutherland, D. R., Nayar, R., Laraya, P., Minden, M., Keating, A., Eaves, A. C., Eaves, C. J., and Dick, J. E., (1998) High level engraftment of NOD/SCID mice by primitive normal and leukemic hematopoietic cells from patients with chronic myeloid leukemia in chronic phase, Blood 91, 2406-2414

NOD/SCID IL2Ry^{null} mice can be obtained from the Jackson Laboratory (Bar Harbor, Me.). Neonate and adult mice are sub-lethally irradiated up to 24 hours prior to transplantation with 100 or 270 rads, respectively, using a Gamma Cell 40 Caesium source as well described in the art. Isolated populations of human cells to be tested for their reconstitution potential (e.g., HSCs that bind the anti-(2-3 sialylated lacto structures) antibodies prepared as described above are then transferred into neonates by intracardiac or face vein injection within the first 48 hours after birth or into adult (2-4 month old) mice by tail vein injection. Secondary transplantation can be performed by transferring 5×10⁶ bone marrow cells from femurs and tibias of the primary-recipient mice into each of three to five lethally irradiated NOG mice. Peripheral blood cells from the secondary-recipient mice are analyzed at 1, 2, 3, 4, and 5 months after transplantation.

At various time points following injection of the test cells into the NOG mice (for example, at 12 weeks to 30 weeks), mice are euthanized by cervical dislocation and blood, bone marrow (tibia, femur), spleen, lymph nodes, and thymus are harvested. These tissues are then subjected to flow cytometric analysis for donor chimerism and leukocyte subsets using methods well known in the art. For example, bone marrow is suspended in DMEM with 0.1% bovine serum albumin. Bone marrow cells are stained with fluorescein isothiocyanate (FITC)-conjugated anti-human CD45 and PE-conjugated anti-mouse CD45; human leukocyte subsets are also stained with one of the following PE-conjugated antibodies: CD3, CD14, CD16, CD20, CD41, and CD56. Red blood cells are stained with anti-mouse TER119-FITC and anti-human glycophorin A (GPA)-PE (CD235a); erythrocyte subsets are stained with human CD45-FITC and CD71-PE. All antibodies can be purchased from BD Biosciences. Alternatively, lineage analysis can be done using antihuman antibodies such as PB-conjugated CD45, HI30; APC-Alexa Fluor 750-conjugated 003, S4.1; APC-conjugated CD19, SJ25-C1; PE-conjugated CD13, TK1 (Caltag); PEconjugated CD33, P67.6, PE-conjugated GPA, GA-R2, APC-conjugated CD41a, HIPS (BD Biosciences). Mouse leukocytes and red cells are identified based on the expression of Alexa488 or PE-Cy7-conjugated CD45.1, clone A20.1.7, and PE-Cy5 or PE-Cy7-conjugated Ter119 (eBiosciences, San Diego, Calif.), respectively. A variety of vendors, including R&D Systems, offer numerous panels of lineage differentiation markers.

Chimerism, or the level of human leukocyte reconstitution, can be calculated as follows, from peripheral blood:

Chimerism=% CD45⁺ human cell/(% CD45⁺ human cell+% CD45⁺ mouse cell)

Using these methods, the identified HSCs with high reconstitution potential will (1) exhibit at least 10% chimerism, or (2) exhibit significant engraftment only after 12 weeks or more in the primary recipient yet show multilineage reconstitution in a secondary recipient.

### Example 6: Ex Vivo Expansion and Differentiation of HSCs with High Reconstitution Potential

HSCs, including primitive early HSCs with high reconstitution potential, can be expanded ex vivo through a variety of methods. Before expansion, HSCs may be genetically modified such that the resulting cells contain the desired genetic modification. Examples of commercially available expansion media and protocols include StemMACS™ HSC Expansion Media (Miltenyi Biotec), STEMGENIX HSC GEM/Stemline™ Medium (SIGMA), and PromoCell's DXF medium (PromoCell GmbH). Other expansion methods are well known in the art. See, for example, Walasek, M A, van Os R, and de Haan G, (2012) Hematopoietic stem cell expansion: challenges and opportunities; Ann N Y, Acad Sci. 2012 August, 1266:138-50; and Rodriguez-Pardo V M and Vernot J P, Mesenchymal stem cells promote a primitive phenotype CD34+c-kit+ in human cord blood-derived hematopoietic stem cells during ex vivo expansion, Cell Mol Biol Lett, 2013 March, 18(1):11-33.

HSCs can differentiate into a variety of lineages. For a review see, for example, Seita J. and Weissman I. L., (2010) Hematopoietic Stem Cell: Self-renewal versus Differentiation, Wiley Interdiscip Rev Syst Biol Med, 2(6): 640-653. Some of these lineages can be differentiated in vitro. For example, a method has been derived to obtain monocytic cells in vitro from BM-derived HSC. Magga J, Savchenko E, Malm T, Rolova T, Pollari E, Valonen P, Lehtonen S̆, Jantunen E, Aarnio J, Lehenkari P, Koistinaho M, Muona A, Koistinaho J, Production of monocytic cells from bone marrow stem cells: therapeutic usage in Alzheimer's disease, J Cell Mol Med, 2012 May, 16(5):1060-73. In addition, stem cells can be cultured in a manner that provides long-lasting precursor cells for bone, cartilage, and lung, Pereira, R. F. et al., Cultured adherent cells from marrow can serve as long-lasting precursor cells for bone, cartilage, and lung in irradiated mice, Proc. Natl. Acad. Sci., USA 92, 4857-4861 (1995). Such culturing can also be conducted with genetically modified stem cells.

What is claimed is:

1. A method for production of an antibody that can be used to identify and isolate human hematopoietic stem cells (HSCs), comprising screening a population of antibodies for an antibody that:
binds to 2-3 sialylated lacto-neolacto type structures; and
can identify human HSCs.

2. The method of claim 1, wherein the population of antibodies is generated against CD34+/CD38− HSCs.

3. The method of any of the preceding claims, wherein the identified HSCs are HSCs with high reconstitution potential.

4. The method of any of the preceding claims, wherein the 2-3 sialylated lacto-neolacto type structures are chosen from sialyl I, sialyl i, sialyllactose, sialyllacto-N-tetraose, sialyllacto-N-neotetraose, and N-acetyl sialyllactoseamine.

5. The method of claim 4, wherein the 2-3 sialylated lacto-neolacto type structure is sialyllactose.

6. The method of any of the preceding claims, wherein the antibody binds to 2-3 sialyated lacto-neolacto type structures on the human stem cell marker CD133.

7. The method of any of the preceding claims, wherein the antibody binds specifically to 3′SL-CD133 and not to neuraminidase-treated 3′SL-CD133.

8. The method any of the preceding claims, wherein the antibody can be used to isolate primitive HSCs with high reconstitution potential as functionally determined by in vivo models.

9. The method of claim 8, wherein the in vivo model is transplantation of primitive HSCs with high reconstitution potential into sub-lethally or lethally-irradiated mice.

10. The method of any of the preceding claims, wherein the antibody binds, or has enhanced binding, to CD133 and human-fucosidase-treated-CD133 but does not bind to neuraminidase-treated CD133.

11. The method of claim 10, wherein the in vivo model is transplantation of primitive HSCs with high reconstitution potential into sub-lethally or lethally-irradiated mice.

12. The method of any of the preceding claims, wherein the HSCs are obtained from at least one source chosen from bone marrow, mobilized peripheral blood, and cord blood.

13. The method of any of the preceding claims, wherein the HSCs are obtained by at least one method chosen from FACS sorting, immunomagnetic beads, and affinity matrices.

14. The method of any of claims 6-13, wherein the human stem cell marker CD133 is isolated from human HSCs or human hematopoietic progenitor cells.

15. A population of HSCs isolated using an antibody produced by the method according to any one of claims 1-14.

16. A population of genetically modified HSCs produced by:
isolating HSCs using an antibody produced by the method according to any one of claims 1-14; and
genetically modifying the isolated HSCs.

17. The population of cells of claim 15 or 16, wherein the cells are isolated from bone marrow, peripheral blood, leukopheresis product, cord blood, or a combination of the same.

18. A population of cells of the lymphoid lineage differentiated from the population of any one of claims 15-17.

19. A population of cells of the erythroid lineage differentiated from the population of any one of claims 15-17.

20. A population of endothelial progenitor cells differentiated from the population of any one of claims 15-17.

21. An isolated mouse monoclonal antibody produced by the method according to any one of claims 1-14.

22. A method of treating a hematologic disease, treating a hematologic disorder, treating a hematologic condition, treating a cardiovascular disorder, treating a wound, rescuing from chemotherapy, and/or rescuing a subject from high-dose radiation comprising administering a population of cells according to any one of claims 15-20.

23. A method of reconstituting hematopoiesis using a population of cells according to any one of claims 15-20.

24. A method of diagnosing a disease, disorder, or condition using an antibody produced according to the method of any one of claims 1-14.

25. A method of purifying HSCs using an antibody produced according to the method of any one of claims 1-14.

26. A method of treating a lymphoma using an antibody produced according to the method of any one of claims 1-14.

27. A method of monitoring a disease, disorder, or condition using an antibody produced according to the method of any one of claims 1-14.

28. A method of monitoring treatment of a disease, disorder, or condition using an antibody produced according to the method of any one of claims 1-14.

29. A method of treating advanced follicular lymphoma using a cell as defined in any one of claims 15-20.

30. A method of treating a pediatric hematologic disease using a cell as defined in any one of claims 15-20.

31. A method of eating a hematologic disorder using a cell as defined in any one of claims 15-20.

32. A method of treating a cardiovascular disorder using a cell as defined in any one of claims 15-20.

33. A method of promoting wound healing using a cell as defined in any one of claims 15-20.

34. The method of claim 30 wherein the disease is acute myeloid leukemia.

35. A method of treating a genetic disease, disorder, or condition using a cell as defined in any one of claims 16-20.

36. A method of treating a patient with a hematologic disease, disorder, or condition comprising:
obtaining a population of cells from a donor;
identifying HSCs in the population of cells using an antibody produced by the method according to any one of claims 1-14; and
administering the identified HSCs or cells derived from the identified HSCs to the patient.

37. A method of treating a patient recovering from chemotherapy or radiation exposure comprising:
obtaining a population of cells from a donor;
identifying HSCs in the population of cells using an antibody produced by the method according to any one of claims 1-14; and
administering the identified HSCs or cells derived from the identified HSCs to a patient recovering from chemotherapy or radiation exposure.

38. A method of promoting wound healing in a patient suffering a wound comprising:
obtaining a population of cells from a donor;
identifying HSCs in the population of cells using an antibody produced by the method according to any one of claims 1-14; and
administering the identified HSCs or cells derived from the identified HSCs to a patient recovering from chemotherapy or radiation exposure.

39. A method of treating a patient with a genetic disease, disorder, or condition comprising:
obtaining a population of cells from a donor;
identifying HSCs in the population of cells using an antibody produced by the method according to any one of claims 1-14;
genetically modifying the identified HSCs; and
administering the genetically modified HSCs or cells derived from the genetically modified HSCs to the patient.

40. The method of any one of claims 36-39, wherein the donor is the patient.

41. The method of any one of claims 36-39, wherein the donor is not the patient.
